(19) United States
(12) Patent Application Publication
(10) Pub. No.: US 2006/0008861 A1
(43) Pub. Date: Jan. 12, 2006

(54) Incorporation of anaerobic bacteria in feed formulation

(76) Inventors: F. C. Thomas Allnutt, Glenwood, MD (US); Moti Harel, Baltimore, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/511,202

(22) PCT Filed: Apr. 14, 2003
(86) PCT No.: PCT/US03/11366

§ 371 Date: Aug. 10, 2005

Related U.S. Application Data
(60) Provisional application No. 60/372,081, filed on Apr. 15, 2002.

(51) Int. Cl. C12Q 1/62

(52) U.S. Cl. 435/10

(57) ABSTRACT

Anaerobic bacteria are incorporated in a feed such that they provide a benefit to the consuming animal. Anaerobic bacteria can be viable and colonize the gut to provide this benefit by displacing harmful bacteria, secreting a particular agent (e.g., enzyme, antibiotic or bioactive compound), binding or sequestering harmful organisms or compounds, or providing a beneficial physical effect. Anaerobic bacteria can also be added n a non-viable form wherein the added bacteria provide a benefit to the consuming organism by delivering preformed compounds such as enzymes, bioactive agents, or polymers. Recombinant anaerobes can be utilized for any of the above purposes. Feeds can contain naked bacteria, spores, treated bacteria such as encapsulated, coated or freeze dried bacteria, in order to maintain either viability or stability of the active function.

### BACKGROUND OF THE INVENTION

This application relates to providing an animal feed containing anaerobic bacteria. It also relates to a method of producing the feed. Finally, it relates to the feed composition itself.

Anaerobic bacteria are known to inhabit the intestines of many animals and to play an important role in microenvironments within the intestines. Some positive influence of anaerobic bacteria is in the secretion of digestive enzymes to aid in the degradation of ingested foodstuffs. Alternatively, anaerobes have been shown to colonize the gut lining and prevent colonization by harmful bacteria.

The presence of anaerobic bacteria in agricultural and aquacultural animals is also well documented. Anaerobic bacteria play a large role in ruminant nutrition and proper balance of ruminant microbial populations is critical for optimal animal production. Less is known about aquacultural systems, however the presence of both facultative and obligate anaerobes has been confirmed.

The importance of recombinant proteins for modern medical applications and therapy cannot be overemphasized. Recombinant production methods for bacteria are well developed [Jonasson, 2002 #627]. Many important commercial proteins are produced in bacterial prokaryotic systems, having importance in industry and medical science.

Aquaculture as an industry is rapidly being developed for production of biomass for food (e.g., shrimp and fish farming) [Halvorson, 1999 #534]. The rapid rise of this industry has led to excessive reliance on antibiotics, both for treatment of disease and as prophylactic additions. This is causing the development of bacterial resistance to the available drugs as well as causing pollution of the aquatic environment. The cost of antibiotics is also a burden on the industry.

Agriculture as an industry also has relied heavily on the application of antibiotics for treatment of disease states and prophylactic applications. However, the addition of subclinical amounts of antibiotic as a growth enhancer has the most potential for producing a damaging effect, by the development of antibiotic resistance. Since many of the antibiotics used for agriculture are related to antibiotics used for human health, agricultural application of antibiotics could eventually impact human antibiotic therapy.

Thus, there is a need for new methods for maintaining the health of animals. This is especially true for agricultural and aquacultural animals.

### SUMMARY OF THE INVENTION

In a first aspect, the invention provides methods of producing a feed containing anaerobic bacteria that provide a benefit to the consuming animal.

In a second aspect, the invention provides a feed containing anaerobic bacteria that imparts a benefit to the consuming animal.

These and other aspects of the invention are provided by one or more of the following embodiments.

In one embodiment, the invention provides the composition itself, a feed comprising an anaerobic bacterium. The feed can be an aquaculture feed, including, but not limited to, a feed for fish or crustaceans. The feed can also be an agriculture feed, including, but not limited to, a feed for chickens.

In another embodiment, the feed contains probiotic elements.

In yet another embodiment, the anaerobic bacterium in the feed is viable. Alternatively, the bacterium can be in the form of a spore. The anaerobic bacterium in the feed can also be non-viable. The invention encompasses mixtures of viable, sporulated, and non-viable bacteria.

In a further embodiment, the anaerobic bacterium can be an obligate anaerobe; alternatively it can be a facultative anaerobe.

Examples of anaerobic bacteria according to the invention are members of the genera Clostridium, Fusobacterium, Peptostreptococcus, Bacteriodes, Butyrivibrio, Leptptrichia, Selenomonas, Succinimonas, Succinivibrio, Eubacterium, Lachnospira, Aracnia, Propionibacterium, Actinomyces, Bifidobacterium, Lactobacillus, Treponema, Borrelia, and Campylobacter.

In another embodiment, the anaerobic bacterium is recombinant. This recombinant bacterium can include an antisense ribonucleic acid. It can also include bioactive compounds, e.g., a protein or peptide. Bioactive peptides of the invention include, but are not limited to, cecropins, penaedins, bactenecins, callinectins, myticins, tachyplesins, clavanins, misgurins, pleurocindins, parasins, histones, acidic proteins, and lysozymes.

In a further embodiment, the animal feed is effective when the concentration of the anaerobic bacterium in the feed is at least about 0.01%. The animal feed is also effective when the concentration of the anaerobic bacterium in the feed is between about 0.01% and 10%, between about 0.01% and 1.0%, and between about 0.01% and 0.1%. Further, the animal feed is effective when the concentration of the anaerobic bacteria in the feed is between about 0.1% and 1.0%.

In one embodiment, the invention provides a method of producing an aquacultural or agricultural feed, wherein the feed comprises an anaerobic bacterium. The feed is produced by mixing the bacterium with animal feed.

In another embodiment, the invention provides a method of producing animal feed that contains an anaerobic bacterium that is added in a potentially viable but stable natural state, e.g., a spore that colonizes the intestines of the consuming animal. The feed is produced by mixing spores with animal feed.

In a further embodiment, the feed of the invention is produced to contain an anaerobic bacterium in a nonviable state, such that compounds beneficial to the animal are released into the intestines of the consuming animal. The feed is produced by rendering the bacteria nonviable, and mixing the bacteria with an animal feed. The bacterium can be dried before it is mixed with animal feed.

In yet a further embodiment, the feed is produced by mixing Clostridium difficile spores with AquaGrow Enhance® (Advanced BioNutrition), either prior to, or subsequent to, the blending of the AquaGrow Enhance® feed.

In another embodiment, the feed is produced by genetically modifying a facultative anaerobe to express a bioactive peptide. Suitable bioactive peptides include, but are not limited to, cecropins, penaedins, bactenecins, callinectins, myticins, tachyplesins, clavanins, misgurins, pleurocindins, parasins, histones, acidic proteins, and lysozymes. The anaerobic bacteria are grown, harvested, and added to animal feed. The bacteria can be mixed with yeast, and/or dried before they are added to the feed.

In yet another embodiment, the feed is produced by growing a biomass of Photobacterium damselae subsp. piscicida under anaerobic conditions, harvesting the bacteria, and rendering them nonviable. The bacteria can be grown on agar. The bacteria are added to animal feed, and upon ingestion, they block adsorption of live P. damselae subsp. piscicida to the intestine of the animal that has consumed the feed. The bacteria can be dried before they are added to the animal feed.

In a further embodiment, the feed is produced by growing a biomass of Clostridium difficile under anaerobic conditions, rendering them nonviable, and cracking the cells in a manner that retains their enzymatic activity. The cracked cells can be dried. They are incorporated into animal feed, and the Clostridium difficile digestive enzymes are transferred to the animal that consumes the feed.

The inventors have discovered that adding viable or nonviable anaerobic bacteria to feeds, provides beneficial effects to the consuming animal.

### DETAILED DESCRIPTION OF THE INVENTION

Definitions

An “anaerobic bacterium” is an organism that can survive without the presence of molecular oxygen.

A “facultative anaerobic bacterium” is a bacterium that can survive with or without the presence of molecular oxygen.

An “obligate anaerobic bacterium” is a bacterium that can only survive in the absence of molecular oxygen.

A “bacterial spore” is a resistant structure or stage formed by prokaryotes that is capable of germination into viable cells.

A “probiont” is any viable organism that is provided to another organism for the purposes of colonization within that organism with a beneficial effect.

“Viable” is a condition wherein the organism can multiply and sustain itself.

“Non-viable” is a condition wherein the organism is incapable of multiplication or sustaining itself.

### EXAMPLES

Certain embodiments of the invention will now be described in more detail through the following examples. The examples are intended solely to aid in more fully describing selected embodiments of the invention and should not be considered to limit the scope of the invention in any way.

### Example 1

### Production of an Aquacultural Feed Containing Anaerobic Bacteria

Clostridium difficile is grown in anaerobic medium and allowed to sporulate using standard anaerobic methods [Holdeman, 1975 #656]. Spores are collected by centrifugation, washed with phosphate buffered saline (autoclaved and purged with nitrogen or inert gas), and mixed with AquaGrow Enhance® (Advanced BioNutrition). This mixing can be done after the AquaGrow Enhance® has been spray dried and blended or prior to this blending. The final material is then available for use as a feed for larval fish and/or crustacean culture.

### Example 2

### Production of a Chicken Feed Containing Anaerobic Bacterium Expressing an Antibiotic Peptide

Using standard techniques [Sambrook, 1989 #109], a facultative anaerobe is genetically modified to express one or more of the following bioactive peptides: cecropin, penaedins, bactenecins, callinectins, myticins, tachyplesins, clavanins, misgurins, pleurocindins, parasins, histones, acidic proteins, and lysozymes. This anaerobe is grown anaerobically to maintain the stability of the peptide in an optimal (non-oxidized form) and the culture harvested by centrifugation. The biomass is mixed with dry yeast and then flash dried either in a spray drier or by vacuum drying. The homogenized powder can then be supplemented into regular chicken feeds to deliver the bioactive compound.

### Example 3

### Anaerobically Grown Photobacterium as an Addition to Aquaculture Feed

Photobacterium damselae subsp. piscicida are grown on blood agar or agar containing hematin under anaerobic conditions between 18 and 22 degrees C. Cells are harvested and killed by heat treatment or by treatment with formaldehyde. Cells are then dried in a spray drier or by use of a vacuum drier. These cells are tested for viability by culturing; if they are non-viable, then they can be added to feeds for use to block adsorption to the gut by live P. damselae subsp. piscicida.

### Example 4

### Clostridium Difficile Killed and Added as a Feed Supplement

Clostridium difficile is grown under anaerobic conditions as previously described [Holdeman, 1975 #656]. The biomass is killed by heat treatment, air-drying, or radiation. Cells are then broken by grinding to crack the cells; the use of a roller mill or Wiley mill provides sufficient cracking. The most gentle methods used provide the best retention of enzyme activity. The dried cells are incorporated into feed to provide maximal transfer of digestive enzymes to the consuming organism.

### REFERENCES

The following references are cited herein. The entire disclosure of each reference is relied upon and incorporated by reference herein.
1. Halvorson, H. and F. Quezada (1999). “Increasing public involvement in enriching our fish stocks through genetic enhancement.” Genet. Anal. 15(3-5): 75-84.
2. Holdeman, L. and W. Moore (1975). Anaerobe laboratory manual.
3. Jonasson, P., S. Liljeqvist, et al. (2002). “Genetic design for facilitated production and recovery of recombinant proteins in Escherichia coli.”Biotechnol Appl. Biochem. 35(Pt 2): 91-105.
4. Sambrook, J., E. Fritsch, et al. (1989) Molecular Cloning: A laboratory manual. Cold Spring Harbor, Cold Spring Harbor Press.

We claim:

1. A method of animal feed production, comprising mixing at least one anaerobic bacterium with the feed.

2. The method of claim 1, wherein the feed is an aquaculture feed.

3. The method of claim 2, wherein the feed is a fish feed.

4. The method of claim 2, wherein the feed is a crustacean feed.

5. The method of claim 1, wherein the feed is an agriculture feed.

6. The method of claim 5, wherein the feed is a chicken feed.

7. The method of claims 1-6, further comprising mixing at least one probiotic element with the feed.

8. The method of claims 1-6, wherein at least one anaerobic bacterium is viable at the time of production.

9. The method of claims 1-7, wherein at least one anaerobic bacterium comprises a spore.

10. The method of claims 8-9, wherein the feed is produced by mixing Clostridium difficile spores with AquaGrow Enhance® feed.

11. The method of claim 10, wherein the Clostridium difficile spores are mixed with the AquaGrow Enhance® feed prior to the blending of the AquaGrow Enhance® feed.

12. The method of claim 10, wherein the Clostridium difflcile spores are mixed with the AquaGrow Enhance® feed subsequent to the blending of the AquaGrow Enhance® feed.

13. The method of claims 1-7, wherein at least one anaerobic bacterium is non-sporulated.

14. The method of claims 1-7, wherein at least one anaerobic bacterium is non-viable at the time of production.

15. The method of claims 1-6 and 14, further comprising growing a biomass of Clostridium difficile under anaerobic conditions, rendering them nonviable, and cracking the Clostridium in a manner that retains enzymatic activity.

16. The method of claims 1-15, wherein at least one anaerobic bacterium is a Clostridium, Fusobacterium, Peptostreptococcus, Bacteriodes, Butyrivibrio, Leptptrichia, Selenomonas, Succinimonas, Succinivibrio, Eubacterium, Lachnospira, Aracnia, Propionibacterium, Actinomyces, Bifidobacterium, Lactobacillus, Treponema, Borrelia, or Campylobacter, or a mixture of two or more of these.

17. The method of claims 1-16, wherein at least one anaerobic bacterium comprises an obligate anaerobe.

18. The method of claims 1-9, 13-14, and 16, wherein at least one anaerobic bacterium comprises a facultative anaerobe.

19. The method of claims 1-18, wherein at least one anaerobic bacterium is recombinant.

20. The method of claim 19, further comprising genetically modifying the facultative anaerobe to express a bioactive peptide.

21. The method of claim 19, wherein at least one anaerobic bacterium comprises a recombinant bioactive compound.

22. The method of claim 19, wherein the anaerobic bacterium comprises an antisense ribonucleic acid.

23. The method of claim 19, wherein at least one anaerobic bacterium comprises a recombinant protein or peptide.

24. The method of claims 19-21, wherein at least one anaerobic bacterium comprises a cecropin, penaedin, bactenecin, callinectin, myticin, tachyplesin, clavanin, misgurin, pleurocindin, parasin, histone, acidic protein, or lysozyme.

25. The method of claim 1, further comprising growing the anaerobic bacterium, harvesting the bacterium, mixing the bacterium with yeast, and drying the bacterium.

26. The method of claim 1, further comprising growing a biomass of Photobacterium damselae subsp. piscicida under anaerobic conditions, harvesting the biomass, rendering the Photobacterium nonviable, and drying the Photobacterium.

27. An animal feed comprising at least about 0.01% anaerobic bacterium.

28. The feed of claim 27, wherein the feed is an aquaculture feed.

29. The feed of claim 28, wherein the feed is a fish feed.

30. The feed of claim 28, wherein the feed is a crustacean feed.

31. The feed of claim 27, wherein the feed is an agriculture feed.

32. The feed of claim 31, wherein the feed is a chicken feed.

33. The feed of claim 27, wherein the feed further comprises one or more probiotic elements.

34. The feed of claim 27, wherein at least one anaerobic bacterium is viable at the time of production.

35. The feed of claim 27, wherein at least one anaerobic bacterium comprises a spore.

36. The feed of claim 27, wherein at least one anaerobic bacterium is non-sporulated.

37. The feed of claim 27, wherein at least one anaerobic bacterium is non-viable at the time of production.

38. The feed of claim 27, wherein at least one anaerobic bacterium is a Clostridium, Fusobacterium, Peptostreptococcus, Bacteriodes, Butyrivibrio, Leptptrichia, Selenomonas, Succinimonas, Succinivibrio, Eubacterium, Lachnospira, Aracnia, Propionibacterium, Actinomyces, Bifidobacterium, Lactobacillus, Treponema, Borrelia, or Campylobacter, or a mixture of two or more of these.

39. The feed of claim 27, wherein the anaerobic bacterium comprises at least one obligate anaerobe.

40. The feed of claim 27, wherein the anaerobic bacterium comprises at least one facultative anaerobe.

41. The feed of claim 27, wherein at least one anaerobic bacterium is recombinant.

42. The feed of claim 41, wherein the recombinant anaerobic bacterium comprises one or more bioactive compound.

43. The feed of claim 42, wherein the recombinant anaerobic bacterium comprises one or more antisense ribonucleic acid.

44. The feed of claim 42, wherein the recombinant anaerobic bacterium comprises one or more recombinant protein or peptide.

45. The feed of claim 44, wherein the recombinant anaerobic bacterium comprises a cecropin, penaedin, bactenecin, callinectin, myticin, tachyplesin, clavanin, misgurin, pleurocindin, parasin, histone, acidic protein, or lysozyme.

46. The feed of claims 27-45, wherein the anaerobic bacterium comprises from about 0.01% to 10% of the feed.

47. The feed of claims 27-45, wherein the anaerobic bacterium comprises from about 0.01% to 1.0% of the feed.

48. The feed of claims 27-45, wherein the anaerobic bacterium comprises from about 0.01% to 0.1% of the feed.

49. The feed of claims 27-45, wherein the anaerobic bacterium comprises from about 0.1% to 1.0% of the feed.
